SEQUENCE LISTING (6 sequences)

SEQ ID NO: 1 - DNA, 720 bp, Artificial sequence
  Synthetic eCFP sequence
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 2 - DNA, 720 bp, Artificial sequence
  Synthetic eYFP sequence
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 3 - DNA, 720 bp, Artificial sequence
  Synthetic eYFP Venus sequence
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagctgatctgcaccaccggcaagctgcccgtgccctggcccaccctcgtgaccaccctgggctacggcctgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 4 - DNA, 675 bp, Artificial sequence
  Synthetic genetically altered eCFP
atggagctgttcaccggggtggtgcccatactggtcgagctggatggcgatgtaaatggccacaaattcagcgtgtccggcgagggcgaaggcgatgccacctacggcaaactgaccctgaaattcatatgcaccaccggcaagctgcccgtcccctggcccaccctcgtgaccaccctgacctggggcgtgcagtgtttcagccgctaccccgatcatatgaagcaacacgatttctttaagtccgccatgcccgaaggctatgtccaagagcgcaccatattctttaaggatgacggcaattacaaaacccgcgccgaggtgaaattcgagggcgacaccctggtgaatcgcattgagctgaaaggcatcgattttaaggaagacggcaatatcctggggcacaaactggagtataactatatcagccacaatgtctatattaccgccgacaaacagaaaaacggcataaaggccaactttaagatacgccacaatatcgaagacggcagcgtgcagctcgccgaccattaccaacagaatacccccatcggcgacggccccgtgctgctgcccgacaatcactatctgagcacccagtccgccctgagcaaagaccccaacgaaaagcgcgatcatatggtcctgctcgaatttgtgaccgccgccgggatc

SEQ ID NO: 5 - DNA, 675 bp, Artificial sequence
  Synthetic genetically altered eYFP Venus
gagttgtttacgggcgtcgtcccgatcctcgtggaactcgacggggatgttaacgggcataagttttcggtcagcggggaaggggagggggacgcgacgtatgggaagctcactctcaagctgatctgtacgacggggaaactcccggtcccgtggccgacgctggtcacgacgctgggatacgggctccaatgctttgcgaggtatccggaccacatgaaacagcatgactttttcaaatcggcgatgccggagggatacgtgcaggaacggacgatctttttcaaagacgatgggaactataagacgcgggcggaagtcaagtttgaaggggacacgctcgtcaaccggatcgaactcaaggggattgacttcaaagaggatgggaacatactcggccataagctcgaatacaattacaactcgcataacgtatacatcaccgcggataagcaaaagaatgggatcaaagccaatttcaaaatccggcataacatagaggatgggggggtccaactggcggatcactatcagcaaaacacgccgataggggatgggccggtcctcctcccggacaaccattacctctcgtaccaaagcgcgctctcgaaggacccgaatgagaaacgggaccacatggttctcctggagttcgtcacggcggcgggcatatag

SEQ ID NO: 6 - DNA, 675 bp, Artificial sequence
  Synthetic genetically altered Venus
gagttgtttacgggcgtcgtcccgatactcgtggaactcgatggggatgttaatgggcataaattttcggtcagcggggaaggggaaggggacgcgacgtatgggaaactcactctgaaactgatatgtacgacggggaaactcccggtcccgtggccgacgctggtcacgacgctgggatacgggctccaatgttttgcgaggtatccggatcatatgaaacaacatgatttttttaaatcggcgatgccggagggatatgtgcaagaacggacgatattttttaaagatgatgggaattataaaacgcgggcggaagtcaaatttgaaggggatacgctcgtcaatcggattgaactcaaagggattgattttaaagaagatgggaatatactcggccataaactcgaatataattataactcgcataatgtatacattaccgcggataaacaaaaaaatgggataaaagcgaattttaaaatacggcataatatagaagatgggggggtccaactggcggatcattatcaacaaaatacgccgataggggatgggccggtcctcctcccggataatcattatctctcgtaccaaagcgcgctctcgaaggacccgaatgaaaaacgggaccatatggttctcctcgaatttgtcacggcggcgggcatatga

(19) United States
(12) Patent Application Publication
(10) Pub. No.: US 2010/0138944 A1
(43) Pub. Date: Jun. 3, 2010

(54) Methods of Reducing Repeat-Induced Silencing of Transgene Expression and Improved Fluorescent Biosensors

(76) Inventors: Wolf B Frommer, Stanford, CA (US); Karen Deuschle, Heidenheim (DE)

Correspondence Address: MORGAN LEWIS & BOCKIUS LLP, 1111 PENNSYLVANIA AVENUE NW, WASHINGTON, DC, 20004, US

(21) Appl. No.: 12/083,197

(22) PCT Filed: Oct. 14, 2005
(86) PCT No.: PCT/US05/36953

§ 371 Date: Aug. 28, 2009

(51) Int. Cl. C12N 15/11; C07H 21/00; C12N 15/31; C12N 5/10; C12N 1/21; C12N 1/19; C12N 15/85; C12N 15/81; C12N 15/82; C12N 15/70; C07K 2/00; C12Q 1/68; A01K 67/027; A01H 5/00

(52) U.S. Cl. 800/13; 536/23.1; 536/23.7; 435/325; 435/419; 435/252.33; 435/254.2; 435/320.1; 800/298; 530/350; 435/6; 435/468; 435/456

(57) ABSTRACT

Methods of avoiding repeat- and homology-induced silencing of transgenes are disclosed, in which transgene sequences are genetically altered to reduce the affects of gene silencing. FRET biosensors containing such genetic alterations for improved expression in cell lines and in vivo are disclosed.

### FIELD OF INVENTION

This invention relates to improved methods of expressing recombinant genetic constructs in cells and whole organisms, and particularly to the design and expression of recombinant genetic constructs that exhibit reduced susceptibility to repeat- or homology-induced silencing of transgene expression.

### BACKGROUND OF INVENTION

Eukaryotic organisms possess a variety of efficient defense systems to guard against the invasion and expression of foreign nucleic acids. These defense systems have recently been recognized as a significant hurdle to gene therapy and other endeavors to express exogenous transgenes in plants and animals. See, e.g., Bestor, 2000, Gene silencing as a threat to the success of gene therapy, J. Clin. Invest. 105(4): 409-11. Although eukaryotic defense mechanisms may be mediated by diverse modes of operation, one common trigger is the presence of repeat DNA in the transgene nucleic acid.

For instance, gene silencing may occur at either the transcriptional or post-transcriptional level, and may accompany methylation of DNA and changes in chromatin structure. One form of transcriptional silencing has been termed “repeat-induced gene silencing” (RIGS), and was described at least thirteen years ago in Arabidopsis. Assaad et al., 1992, Somatic and germinal recombination of a direct repeat in Arabidopsis, Genetics 132(2): 553-66. RIGS is strictly dependent on the presence of repeated DNA sequences, and is correlated with the absence of steady state mRNA, increased methylation of DNA and increased resistance of DNA to enzymatic digestion. These observations led Ye and Signer to postulate that repeated nucleotide sequences lead chromatin to adopt a local configuration that is difficult to transcribe, similar to heterochromatin formation. Ye and Signer, 1996, RIGS (repeat-induced gene silencing) in Arabidopsis is transcriptional and alters chromatin configuration.

More recently, RIGS has been described in other eukaryotic organisms and is now thought to be a universal silencing mechanism. Henikoff, 1998, Conspiracy of silence among repeated transgenes, Bioessays 20(7): 532-5. For instance, it has also been reported that DNA methylation and changes in chromatin structure are associated with RIGS in the fungus Neurospora crassa. Meyer, 1996, Repeat-induced gene silencing: common mechanisms in plants and fungi, Biol. Chem. Hoppe Seyler 377(2): 87-95. Garrick and colleagues reported that a reduction of transgene copy number in transgenic mouse lines resulted in a marked increase in transgene expression accompanied by decreased chromatin compaction and decreased methylation at the transgene locus. Garrick et al., 1998, Repeat-induced gene silencing in mammals, Nat. Genet. 18(1): 5-6. In addition, it was reported that inhibitors of histone deacetylase decrease the silencing of multicopy transgenes in murine embryonal carcinoma stem cells, suggesting that RIGS is at least one mechanism responsible for triggering silencing in mammalian cells in vitro. McBurney et al., 2002, Evidence for repeat-induced gene silencing in cultured mammalian cells: inactivation of tandem repeats of transfected genes, Exp. Cell Res. 274(1): 1-8. Although RIGS is associated with methylation in most cases, repeat transgenes are also subject to silencing in Drosophila melanogaster, which exhibits no detectable modified DNA. Dorer and Henikoff, 1997, Transgene repeat arrays interact with distant heterochromatin and cause silencing in cis and trans, Genetics 147: 1181-1190. Accordingly, methylation-independent mechanisms of RIGS may also exist.

RIGS has also been called “transcriptional cis-inactivation” in plants because silencing is observed between neighboring repeated sequences and transgene arrays. However, transcriptional gene silencing (TGS) of transgenes can also occur in trans, both by a paramutation-like mechanism and by ectopic trans-inactivation. Vaucheret et al., 1998, Transgene-induced gene silencing in plants, Plant J. 16(6): 651. Paramutation is actually a natural epigenetic phenomenon where a host gene can become silent and methylated when brought into the presence of a silenced homologous copy, and can acquire the ability to inactivate other copies in subsequent crosses. Vaucheret at al., 1998; Meyer et al., 1993, Differences in DNA methylation are associated with a paramutation phenomenon in transgenic petunia, Plant J. 4:89-100. The mechanism is thought to involve DNA-DNA pairing and transmission of chromatin structure from the silent copy to the inactive copy, as shown in Drosophila with the transmission of position-effect variegation (PEV). Vaucheret at al., 1998; Karpen, 1994, Position-effect variegation and the new biology of heterochromatin, Curr. Opin. Genetic Dev. 4: 281-91.

Ectopic trans-inactivation differs from paramutation in that active transgenes are silenced when brought into the presence of an unlinked silenced homologous transgene, but do not acquire the ability to inactivate in trans other unlinked transgenes. Vaucheret at al., 1998; Matzke et al., 1989, Reversible methylation and inactivation of marker genes in sequentially transformed tobacco plants, EMBO J. 8: 643-49. Deletion analysis has indicated that 90 base pairs of homology in the promoter region of transgenes is sufficient for this type of silencing, indicating that homologous promoter regions may be one target for this phenomenon. Thierry and Vaucheret, 1996, Sequence homology requirements for transcriptional silencing of 35S transgenes and post-transcriptional silencing of nitrate reductase (trans) genes by the tobacco 271 locus, Plant Mol. Biol. 32: 1075-83. Possible mechanisms for ectopic trans-inactivation include direct DNA pairing between a stably integrated transgene and another gene with a homologous promoter at a separate location of the genome. Vaucheret, 1998. Another possible mechanism could be the production of a diffusible RNA that leads to methylation and silencing of the homologous locus via an RNA-DNA interaction. Vaucheret at al., 1994, Promoter dependent trans-inactivation in transgenic tobacco plants: kinetic aspects of gene silencing and gene reactivation, C.R. Acad. Sci. Paris 317:310-23; Park et al., 1996, Gene silencing mediated by promoter homology occurs at the level of transcription and results in meiotically heritable alterations in methylation and gene activity, Plant J. 9: 183-94; Wassenegger and Pelissier, 1998, A model for RNA-mediated gene silencing in higher plants, Plant Mol. Biol. 37: 349-62.

As noted above, gene silencing as a result of repeated DNA can also occur at the post-transcriptional level, i.e., when RNA does not accumulate even in the presence of transcription. For instance, as reported by Ma and Mitra, transgenes with intrinsic direct repeats induced post-transcriptional gene silencing at a very high frequency in transgenic tobacco plants. Ma and Mitra, 2002, Intrinsic direct repeats generate consistent post-transcriptional gene silencing in tobacco, Plant J. 31(1): 37-49. Others have shown that post-transcriptional silencing of nonviral transgenes in transgenic plants prevents subsequent virus infection when homology exists between transgene and viral sequences. English et al., 1996, Suppression of virus accumulation in transgenic plants exhibiting silencing of nuclear genes, Plant Cell 8(2): 179-88. In the fungus N. crassa, repeat-induced point mutation (RIP) leads to both an increase in DNA methylation and degradation of mRNA transcripts expressed from RIP regions. Galagan and Selker, 2004, RIP: the evolutionary cost of genome defense, Trends Genet. 20(9): 417-23; Chicas et al., 2004, RNAi-dependent and RNAi-independent mechanisms contribute to the silencing of RIPed sequences in N. crassa, Nucleic Acids Res. 32(14): 4237-43.

Post-transcriptional gene silencing (PTGS) was originally discovered as the coordinated silencing of transgenes and homologous host genes in plants, which was referred to as “co-suppression.” Napoli et al., 1990, Introduction of a chimeric chalcone synthesis gene into petunia results in reversible co-suppression of homologous genes in trans, Plant Cell 2(4): 279-89. Since then, numerous transgenes encoding part or all of the entire transcribed sequence of a plant host gene have been shown to trigger co-suppression of homologous host genes. Depicker and van Montagu, 1997, Post-transcriptional gene silencing in plants, Curr. Opinion Cell Biol. 9: 373-382. Co-suppression is commonly associated with strongly expressed transgenes, suggesting a mechanism related to aberrant levels of RNA or multiple gene copy number. See, e.g., Lehtenberg et al., 2003, Neither inverted repeat T-DNA configurations nor arrangements of tandemly repeated transgenes are sufficient to trigger transgene silencing, Plant J. 34(4): 507-17. However, PTGS of host gene expression has also been observed in the presence of weakly transcribed or promoterless transgenes, implying that DNA-DNA pairing could play a role in co-suppression. Vaucherot et al., 1998; van Blokland et al., 1994, Transgene-mediated suppression of chalcone synthase expression in Petunia hybrida results in an increase in RNA turnover, Plant J. 6: 861-77.

More recently, a potent form of PTGS termed RNA interference (RNAi) has been discovered. RNAi was first described in the invertebrate organism Caenorhabditis elegans, but is now known to occur in a wide variety of eukaryotic organisms including fruit flies, zebra fish and mammals. Fire et al., 1998, Potent and specific genetic interference by double-stranded RNA in C. elegans, Nature 391: 806-11. The mechanism of RNAi has been widely studied and involves the formation of a double stranded RNA (dsRNA) with homology to a host gene, which is cleaved into small interfering RNA (siRNA) molecules that trigger the degradation of homologous host RNAs in the cytoplasm as wells as the de novo methylation of homologous DNA in the nucleus. Jana et al., 2004, Mechanisms and roles of the RNA-based gene silencing, Elec. J. Biotechnol. 7(3); Matzke and Birchler, 2005, RNAi-mediated pathways in the nucleus, Nat. Rev. Genet. 6(1): 24-35.

Many researchers and companies have harnessed the specificity and potency of RNAi to develop dsRNA-based therapeutics for silencing disease genes and inhibiting virus expression and replication. However, very few researchers have focused on the obstacle that gene silencing mechanisms can present for gene therapy and expression of heterologous genes in cells and whole organisms. U.S. Pat. No. 6,635,806 describes the use of promoters, enhancers, coding sequences and terminators from an alternative plant species to avoid homology-based gene silencing in transgenic maize. US 20050191723 describes the use of Stabilizing Anti-Repressor (STAR™) sequences for the expression of multiple transgenes. STAR™ sequences are described as DNA elements with gene transcription modulating activity that protect transgenes from gene silencing, and particularly RIGS. Finally, US 20031057715 describes the use of low molecular weight, DNA-specific compounds that bind to chromatin-responsive elements (CRE), permitting chromatin remodeling and reduction of gene silencing in Drosophila. What is needed is a universally applicable, straightforward method of improving transgene structure to reduce or circumvent any repeat-driven gene silencing mechanism in any organism.

### SUMMARY OF INVENTION

The present invention provides a solution to the interference by host gene silencing mechanisms in the expression of homologous or heterologous genes or transgenes in a cell or whole organism. In particular, the present invention provides methods of reducing gene silencing of one or more transgenes in a cell, comprising introducing at least one genetic alteration into said one or more transgenes such that the level of identity in at least one repeat or homologous region of said one or more transgenes is reduced, and transfecting said one or more transgenes into said cell, wherein gene silencing of said one or more transgenes is there by reduced. The methods are applicable to reduce any type of gene silencing triggered by the presence of repeat DNA, including but not limited to repeat-induced gene silencing (RIGS), repeat-induced point mutation (RIP), paramutation, ectopic trans-inactivation, co-suppression and RNA interference. The methods are also applicable where the repeat or homologous regions are present in a single transgene, in two or more different transgenes, and where the repeat or homologous regions are present in both the transgene and the DNA of the host cell.

The methods of the present invention are applicable to a wide variety of transgenes. For instance, the methods may be used in instances where the transgene to be expressed exhibits a high level of identity with a host gene, or where the transgene contains a domain or a stretch of bases exhibiting a high level of identity with a part of a host gene. The invention may be used to more efficiently express single transgenes encoding artificial single chain dimers produced by fusion of two monomer sequences with a high level of identity. The methods may also be used to express single transgenes encoding proteins with duplicated domains, e.g., ABC transporters, and for the expression of two or more different transgenes encoding proteins with substantially similar domains.

In particular, the present inventors have found that the methods of the present invention are useful to increase the expression and efficacy of ligand binding fluorescent indicators, or biosensors, which comprise a ligand binding protein moiety, a donor fluorophore moiety fused to the ligand binding protein moiety, and an acceptor fluorophore moiety fused to the ligand binding protein moiety. Because the two fluorophores of many biosensors are derived from the same fluorophore gene and exhibit a high level of identity, the present inventors have found that gene silencing may significantly affect the expression of such biosensors in whole organisms and particularly plants. By reducing the identity between the fluorophore sequences of biosensors, the present inventors have found that expression of such fluorophores may be significantly enhanced.

Accordingly, in one embodiment, among others, the present invention provides an isolated nucleic acid which encodes a ligand binding fluorescent indicator and methods of using the same, the indicator comprising a ligand binding protein moiety, a donor fluorophore moiety fused to the ligand binding protein moiety, and an acceptor fluorophore moiety fused to the ligand binding protein moiety, wherein fluorescence resonance energy transfer (FRET) between the donor moiety and the acceptor moiety is altered when the donor moiety is excited and said ligand binds to the ligand binding protein moiety, and wherein the nucleic acid sequence encoding at least one of either said donor fluorophore moiety or said acceptor fluorophore moiety has been genetically altered to reduce the level of nucleic acid sequence identity between the nucleic acid encoding the donor fluorophore moiety and the nucleic acid encoding the acceptor fluorophore moiety. In the methods of the invention, either one or both of fluorophore sequences may be genetically altered to reduce the level of nucleic acid sequence identity.

A variety of genetic alterations may be used in the methods of the invention, including but not limited to base changes encoding conservative amino acid substitutions and degenerate substitutions at wobble positions of the donor or acceptor fluorophore coding sequence. However, mutations that alter the emission or absorption spectra of the donor and acceptor fluorophore moieties are excluded, as are alterations that adversely affect the activity of the biosensor.

Due to decreased interference from gene silencing, the biosensors of the invention may demonstrate enhanced function in vivo upon expression of the genetically altered, encoding nucleic acid as compared to the same or similar biosensor expressed from a nucleic acid not containing the genetic alterations.

### BRIEF DESCRIPTION OF THE DRAWINGS

FIG. 1 shows a schematic drawing of a FLIP biosensor gene construct.

FIGS. 2A and 2B provide alignments showing the degree of homology between eCFP (SEQ ID NO: 1) and eYFP (SEQ ID NO: 2), and eCFP and eYFP Venus (SEQ ID NO: 3), respectively.

FIG. 3 is a diagram showing the FLIPgludelta13 construct used for transformation of Arabidopsis.

FIG. 4 is a graph showing the change in fluorescence intensity over time in epidermal Arabidopsis cells of a five week old rdr6-11 plant expressing FLIPglu600μdelta13 in response to glucose. +glc indicates the external application of 50 mM glucose. −glc indicates the removal of external glucose. Perfusion was performed in NaPO₄ buffer, pH 7.

FIGS. 5A and 5B provide alignments showing the degree of homology between Ares (SEQ ID NO: 4) (genetically altered eCFP) and Aphrodite (SEQ ID NO: 5) (genetically altered Venus), and eCFP and Aphrodite, respectively. FIG. 5C provides an alignment showing the degree of homology between eCFP (SEQ ID NO: 1) and Mars (SEQ ID NO: 6) (genetically altered Venus).

FIG. 6 is a photograph showing transient expression of FLIPglu600μdelta11 or delta13 in epidermal cells of Nicotiana benthamiana, and YFP fluorescence after excitation of YFP. A: eCFP and eYFP as FRET pair. B: delta11, with eCFP and Aphrodite encoding Venus as FRET pair. C: delta13, with eCFP and Aphrodite encoding Venus as FRET pair.

### DETAILED DESCRIPTION

### Methods of Reducing Gene Silencing of Transgenes

As described above, the present invention provides methods of reducing gene silencing of one or more transgenes in a cell, comprising introducing at least one genetic alteration into said one or more transgenes such that the level of identity or homology in at least one repeat or homologous region of said one or more transgenes is reduced, and transfecting said one or more transgenes into said cell, wherein gene silencing of said one or more transgenes is there by reduced.

As used herein, the phrase “gene silencing” is meant to encompass any form of gene silencing, occurring at either the transcriptional or post-transcriptional level, and including but not limited to repeat-induced gene silencing (RIGS), repeat-induced point mutation (RIP), paramutation, ectopic trans-inactivation, co-suppression and RNA interference. Given that a common mechanism among different forms of gene silencing is the presence of repeat or homologous regions of DNA, “gene silencing” may also be referred to as “repeat- or homology-induced silencing of gene expression or transgene expression,” or alternatively, “repeat- or homology-driven or -associated transgene silencing.” These alternative phrases are not to be confused with the specific phrase “repeat-induced gene silencing” or “RIGS,” which refers to a specific type of transcriptional gene silencing involving changes in chromatin structure and in some cases increased methylation. These alternative phrases are also not to be confused with “homology-induced gene silencing,” which is an art-recognized phrase used interchangeably with the term “co-suppression,” i.e., where introduction of an exogenous gene showing homology with an endogenous host gene leads to post-transcriptional gene silencing of both the exogenous and endogenous gene.

In the context of the present invention, the term “repeat” is used to refer to a sequence of DNA that is identical with another sequence of DNA. The term “homology or “homologous” is used to refer to a sequence of DNA having sufficient identity with another sequence of DNA so as to result in a decrease in gene expression due to transcriptional or post-transcriptional gene silencing. Such regions may be present within a single transgene, in one or more transgenes, or in one or more transgenes when compared to the host genome. The presence of such regions in a transgene may be detected by an increase in transgene expression when the transgene is expressed in a host cell that is deficient in one or more forms of gene silencing as described herein.

“Repeat” and “homologous” regions according to the invention may be any length that is sufficient to result in gene silencing, but are typically at least 10, at least 15, at least 20, at least 25, at least 30, at least 40, at least 50, at least 75, at least 100 or at least 200 bases in length. “Homologous” regions are at least 50%, at least 60%, at least 70%, at least 75%, at least 80%, at least 85%, at least 90%, at least 95%, at least 98%, or at least 99% identical. Since gene silencing in some cases involves small double-stranded RNAs derived from the respective gene with sizes ranging between 21 and 28 base pairs, a repeat in such embodiments includes sequences of at least 21 bases with up to three mismatches in the preferred case, or up to two mismatches in a less preferred case or one mismatch in a less preferred case.

“Gene silencing” is meant to refer to any decrease in the level of gene expression, or the level of RNA or protein produced from an expressed gene, as a result of the presence of repeat or homologous regions of DNA. As such, methods of “reducing” or “decreasing” gene silencing are meant to refer to any method in which gene silencing is reduced or decreased but not necessarily eliminated or inhibited. Methods of eliminating or inhibiting gene silencing using the methods described herein are also included. A decrease in gene silencing may be detected by measuring mRNA levels or protein levels resulting from the disclosed methods of the invention as compared to mRNA or protein levels in the same host cell or organism in the absence of the methods of the invention.

As used herein, the term “transgene” refers to any isolated “exogenous” gene to be expressed recombinantly in a host cell or whole organism, in contrast to “endogenous” genes that are expressed from the host cell genome. Transgenes include “heterologous” genes, which are genes from the genome of one organism that are placed into a different organism or cell of a different organism. Transgenes also include exogenous genes originating from the same organism as the host cell or host organism, for instance, that have been mutated or placed under different regulatory sequences than the endogenous gene such that they take on a different function or expression characteristic. It is also possible to introduce an exogenous gene originating from the host cell or organism into the host for the purpose of complementing a defective endogenous gene or increasing the copy number or expression level of a similar endogenous gene. The term “gene” is meant to include not only the protein coding portion of a nucleic acid, but also the promoter region and any upstream and downstream regulatory regions involved in expression of the gene, including transcription and translation.

The methods of the invention include the use of any genetic alteration to a repeat or homologous region of a gene involved in gene silencing with the purpose of reducing gene silencing and increasing gene expression, including but not limited to substitutions, insertions and deletions, so long as the genetic alteration reduces gene silencing, increases gene expression, and does not adversely affect the function of the protein encoded by the transgene. Such alterations include genetic modifications of the upstream and downstream regulatory regions of a transgene. Such alterations also include those encoding conservative amino acid substitutions in the transgene coding sequence.

Conservative amino acid substitutions are generally defined as amino acid replacements that preserve the structure and functional properties of proteins. The chemical properties of amino acids that permit one to be conservatively substituted for another are well known by those of skill in the art. For instance, hydrophobic amino acids include methionine, alanine, valine, leucine, isoleucine and norleucine. Neutral and hydrophilic amino acids include cysteine, serine and threonine. Acidic amino acids include aspartate and glutamate. Basic amino acids include asparagine, glutamine, histidine, lysine and arginine. Aromatic amino acids include tryptophan, tyrosine and phenylalanine. Glycine and proline are two amino acids that can influence chain orientation and bending.

In one embodiment of the invention, degenerate substitutions may be made at one or more wobble positions of the transgene. Such substitutions are preferred because they change the nucleic acid coding sequence of the transgene without changing the encoded amino acid sequence. The term “wobble” is an art-recognized term that refers to reduced constraint at a position of an anticodon of tRNA that allows alignment of the tRNA with several possible codons. This redundancy is typically seen at the third codon position, for example, both GAA and GAG code for the amino acid glutamine. This property of the genetic code makes it more tolerant of mutations. For instance, four-fold degenerate codons can tolerate any mutation at the third position. Two-fold degenerate codons can tolerate one out of the three base substitutions at the third position. The following table shows the most popular twenty amino acids and the codons that code for each amino acid.

TABLE 1
Amino Acids and Corresponding Codons

| Amino Acid | Abbreviation | Corresponding Codons |
|---|---|---|
| Alanine | A | GCU, GCC, GCG, GCA |
| Arginine | R | AGA, AGG, CGU, CGG, CGC, CGA |
| Asparagine | N | AAU, AAC |
| Aspartic Acid | D | GAU, GAC |
| Cysteine | C | UGU, UGC |
| Glutamine | Q | CAA, CAG |
| Glutamic Acid | E | GAA, GAG |
| Glycine | G | GGU, GGC, GGA, GGG |
| Histidine | H | CAU, CAC |
| Isoleucine | I | AUU, AUC, AUA |
| Leucine | L | UUA, UUG, CUU, CUC, CUG, CUA |
| Lysine | K | AAA, AAG |
| Methionine | M | AUG |
| Phenylalanine | F | UUU, UUC |
| Proline | P | CCU, CCA, CCC, CCG |
| Serine | S | AGU, AGC, UCC, UCU, UCA, UCG |
| Threonine | T | ACU, ACA, ACC, ACG |
| Tryptophan | W | UGG |
| Tyrosine | Y | UAU, UAC |
| Valine | V | GUG, GUC, GUA, GUU |
| Start | | AUG, GUG |
| Stop | | UAG, UGA, UAA |

In the methods of the present invention, any number of genetic alterations may be made in a transgene in order to alter the level of identity between repeat or homologous sequences. Where repeat or homologous sequences exist between two transgenes, different alterations may be made in each transgene sequence to further decrease the level of identity between the two sequences. For instance, in the methods of the invention, at least two, at least five, at least ten, at least fifteen, at least twenty, at least thirty, at least fifty, or at least one hundred degenerate substitutions may be made at the wobble positions of each transgene involved in the gene silencing.

In designing genetic substitutions for the methods of the present invention, the skilled artisan may chose to consider any codon bias present in the host cell or organism in order to further optimize expression. For example, G and C ending codons have been found to be most prevalent in monocot plant species as well as Drosophila. Kawabe and Miyashita, 2003, Patterns of codon usage bias in three dicot and four monocot plant species, Genes Genet. Syst. 78(5): 343-52. In Arabidopsis, codon usage has been associated with gene function, with G/C biased codon usage seen in photosynthetic and housekeeping genes, and A/T biased codon usage found in tissue-specific and stress-induced genes. Chiapello et al., 1998, Codon usage and gene function are related in sequences of Arabidopsis thaliana, Gene 209(1-2): GC1-GC38. In humans, codon usage preference has been shown to vary according to distance from RNA splice sites. Willie and Majewski, 2004, Evidence for codon bias selection at the pre-mRNA level in eukaryotes, Trends Genet. 20(11): 534-38. And organisms with a high metabolic rate contain protein encoding genes with more A-ending codons and have a higher A content in their introns than do organisms with a low metabolic rate. Xia, 1996, Maximizing transcription efficiency causes codon usage bias, Genetics 144(3): 1309-20.

As described above, preferred genetic alterations will result in a modified coding sequence but no changes in amino acid sequence. Where genetic alterations do produce a transgenic protein having one or more conservative substitutions, or insertions or deletions that do not adversely affect protein function, such isolated proteins are also included in the present invention. Vectors, prokaryotic and eukaryotic host cells and transgenic organisms comprising the improved nucleic acids of the invention are also included.

The methods of the present invention will find use in a wide variety of eukaryotic cells and organisms where gene silencing results is a reduction in transgene expression, including plants, animals and fungi. For instance, the methods of the invention may be used to express single transgenes in cells and organisms containing one or more host genes with regions containing repeat or homologous regions as compared to the transgene sequence, or in methods of expressing two or more transgenes from the same or different construct having regions of sequence similarity, e.g., two members of the same gene family. The methods of the invention may be used to reduce gene silencing of single transgenes encoding artificial single chain dimers, e.g., single chain hormones or other glycoproteins that naturally exist as homodimers but have been recombinantly fused perhaps with the intent of introducing a functional mutation in one of the monomers. The methods of the present invention may also be used for the expression of transgenes encoding proteins with duplicated domains, for example, ABC transporters (van der Heide and Poolman, 2002, ABC transporters: one, two or four extracytoplasmic substrate binding sites, EMBO Rep. 3(10): 938-43), beta-propeller domain/kelch repeat-containing proteins (Prag and Adams, 2003, Molecular phylogeny of the kelch-repeat superfamily reveals an expansion of BTB/kelch proteins in animals, BMC Bioinformatics 4: 42), and thrombospondin repeat-containing proteins to name a few (Meiniel et al., 2003, The thrombospondin type 1 repeat (TSR) and neuronal differentiation: roles of SCO-spondin oligopeptides on neuronal cell types and cell lines, Int. Rev. Cytol. 230: 1-39).

In one embodiment, the methods of the invention may be used to enhance the expression of biosensor transgenes in a host cell or organism, as well as the simultaneous expression of more than one fluorescent biosensor in one cell. More broadly, the methods of the invention may also be employed with any use of FRET employing GFP variants, for example in the detection of protein interactions.

Biosensors

As mentioned above, the present inventors have surprisingly found that the methods of the present invention are useful to increase the expression and efficacy of ligand binding fluorescent indicators, or FRET-based biosensors. Exemplary biosensors are described in provisional application Ser. No. 60/643,576, provisional application Ser. No. 60/658,141, provisional application Ser. No. 60/658,142, provisional application Ser. No. 60/657,702, PCT application [Attorney Docket No. 056100-5053, “Phosphate Biosensors and Methods of Using the Same”], and PCT application [Attorney Docket No. 056100-5055, “Sucrose Biosensors and Methods of Using the Same], which are herein incorporated by reference in their entireties. Such biosensors comprise a ligand binding protein moiety, a donor fluorophore moiety fused to the ligand binding protein moiety, and an acceptor fluorophore moiety fused to the ligand binding protein moiety. Because the two fluorophores of many biosensors are derived from the same fluorophore gene and exhibit a high level of identity, the present inventors have found that gene silencing may significantly affect the expression of such biosensors in whole organisms and particularly plants. By reducing the identity between the fluorophore sequences of biosensors, the present inventors have found that expression of the biosensors in a cell or organism may be significantly enhanced.

Accordingly, in one embodiment, among others, the present invention provides an isolated nucleic acid which encodes a ligand binding fluorescent indicator and methods of using the same, the indicator comprising a ligand binding protein moiety, a donor fluorophore moiety fused to the ligand binding protein moiety, and an acceptor fluorophore moiety fused to the ligand binding protein moiety, wherein fluorescence resonance energy transfer (FRET) between the donor moiety and the acceptor moiety is altered when the donor moiety is excited and said ligand binds to the ligand binding protein moiety, and wherein the nucleic acid sequence encoding at least one of either said donor fluorophore moiety or said acceptor fluorophore moiety has been genetically altered to reduce the level of nucleic acid sequence identity between the nucleic acid encoding the donor fluorophore moiety and the nucleic acid encoding the acceptor fluorophore moiety in order to reduce gene silencing of the indicator transgene.

In the methods of the invention, either one or both of fluorophore sequences may be genetically altered to reduce the level of nucleic acid sequence identity. The fluorophore coding sequences may be fused to the termini of the ligand binding domain. Alternatively, either or both of the donor fluorophore and/or said acceptor fluorophore moieties may be fused to the ligand binding protein moiety at an internal site of said ligand binding protein moiety. Such fusions are described in provisional application No. 60/658,141, which is herein incorporated by reference. Preferably, the donor and acceptor moieties are not fused in tandem, although the donor and acceptor moieties may be contained on the same protein domain or lobe. A domain is a portion of a protein that performs a particular function and is typically at least about 40 to about 50 amino acids in length. There may be several protein domains contained in a single protein.

A “ligand binding protein moiety” according to the present invention can be a complete, naturally occurring protein sequence, or at least the ligand binding portion or portions thereof. In preferred embodiments, among others, a ligand binding moiety of the invention is at least about 40 to about 50 amino acids in length, or at least about 50 to about 100 amino acids in length, or more than about 100 amino acids in length.

Preferred ligand binding protein moieties according to the present invention, among others, are transporter proteins and ligand binding sequences thereof, for instance transporters selected from the group consisting of channels, uniporters, coporters and antiporters. Also preferred are periplasmic binding proteins (PBP), such as any of the bacterial PBPs included in Table 2 below. Bacterial PBPs comprise two globular domains or lobes and are convenient scaffolds for designing FRET sensors. Fehr et al., 2003, J. Biol. Chem. 278: 19127-33. The binding site is located in the cleft between the domains, and upon binding, the two domains engulf the substrate and undergo a hinge-twist motion. Quiocho and Ledvina, 1996, Mol. Microbiol. 20: 17-25. In type I PBPs, such as GGBP (D-GalactoseD-Glucose Binding Protein), the termini are located at the proximal ends of the two lobes that move apart upon ligand binding. Fehr et al., 2004, Current Opinion in Plant Biology 7: 345-51. In type II PBPs, such as Maltose Binding Protein (MBP), the termini are located at the distal ends of the lobes relative to the hinge region and come closer together upon ligand binding. Thus, depending on the type of PBP and/or the position of the fused donor or acceptor moiety, FRET may increase or decrease upon ligand binding and both instances are included in the present invention.

TABLE 2
Bacterial Periplasmic Binding Proteins

| Gene name | Substrate | Species | 3D | Reference |
|---|---|---|---|---|
| AccA | agrocinopine | Agrobacterium sp. | —/— | J. Bacteriol. (1997) 179, 7559-7572 |
| AgpE | alpha-glucosides (sucrose, maltose, | Rhizobium meliloti | —/— | J. Bacteriol. (1999) 181, 4176-4184 |
| | trehalose) | | | |
| AlgQ2 | Alginate | Sphingomonassp. | —/c | J. Biol. Chem. (2003) 278, 6552-6559 |
| AlsB | Allose | E. coli | —/c | J. Bacteriol. (1997) 179, 7631-7637 |
| | | | | J. Mol. Biol. (1999) 286, 1519-1531 |
| AraF | Arabinose | E. coli | —/c | J. Mol. Biol. (1987) 197, 37-46 |
| | | | | J. Biol. Chem. (1981) 256, 13213-13217 |
| AraS | Arabinose/fructose/xylose | Sulfolobus solfataricus | —/— | Mol. Microbiol. (2001) 39, 1494-1503 |
| ArgT | lysine/arginine/ornithine | Salmonella typhimurium | o/c | Proc. Natl. Acad. Sci. USA (1981) 78, |
| | | | | 6038-6042 |
| | | | | J. Biol. Chem. (1993) 268, 11348-11355 |
| ArtI | Arginine | E. coli | | Mol. Microbiol. (1995) 17, 675-686 |
| ArtJ | Arginine | E. coli | | Mol. Microbiol. (1995) 17, 675-686 |
| b1310 | (putative, multiple sugar) | E. coli | —/— | NCBI accession A64880 |
| b1487 | (putative, oligopeptide binding) | E. coli | —/— | NCBI accession B64902 |
| b1516 | (sugar binding protein homolog) | E. coli | —/— | NCBI accession G64905 |
| BtuF | vitamin B12 | E. coli | —/— | J. Bacteriol. (1986) 167, 928-934 |
| CAC1474 | proline/glycine/betaine | Clostridium acetobutylicum | —/— | NCBI accession AAK79442 |
| Cbt | dicarboxylate | E. coli | —/— | J. Supramol. Struct. (1977) 7, 463-80 |
| | (succinate, malate, fumarat) | | | J. Biol. Chem. (1978) 253, 7826-7831 |
| | | | | J. Biol. Chem. (1975) 250, 1600-1602 |
| CbtA | Cellobiose | Sulfoblobus solfataricus | —/— | Mol. Microbiol. (2001) 39, 1494-1503 |
| ChvE | Sugar | Agrobacterium | —/— | J. Bacteriol. (1990) 172, 1814-1822 |
| | | tumefaciens | | |
| CysP | thiosulfate | E. coli | —/— | J. Bacteriol. (1990) 172, 3358-3366 |
| DctP | C4-dicarboxylate | Rhodobacter capsulatus | —/— | Mol. Microbiol. (1991) 5, 3055-3062 |
| DppA | dipeptides | E. coli | o/c | Biochemistry (1995) 34, 16585-16595 |
| FbpA | Iron | Neisseria gonorrhoeae | —/c | J. Bacteriol. (1996) 178, 2145-2149 |
| FecB | Fe(III)-dicitrate | E. coli | | J. Bacteriol. (1989) 171, 2626-2633 |
| FepB | enterobactin-Fe | E. coli | —/— | J. Bacteriol. (1989) 171, 5443-5451 |
| | | | | Microbiology (1995) 141, 1647-1654 |
| FhuD | ferrichydroxamate | E. coli | —/c | Mol. Gen. Genet. (1987) 209, 49-55 |
| | | | | Nat. Struct. Biol. (2000) 7, 287-291 |
| | | | | Mol. Gen. Genet. (1987) 209, 49-55 |
| FliY | Cystine | E. coli | —/— | J. Bacteriol. (1996) 178, 24-34 |
| | | | | NCBI accession P39174 |
| GlcS | glucose/galactose/mannose | Sulfolobus solfataricus | —/— | Mol. Microbiol. (2001) 39, 1494-1503 |
| GlnH | Glutamine | E. coli | o/— | Mol. Gen. Genet. (1986) 205, 260-9 |
| (protein: | | | | J. Mol. Biol. (1996) 262, 225-242 |
| GLNBP) | | | | J. Mol. Biol. (1998) 278, 219-229 |
| GntX | Gluconate | E. coli | —/— | J. Basic. Microbiol. (1998) 38, 395-404 |
| HemT | Haemin | Yersinia enterocolitica | —/— | Mol. Microbiol. (1994) 13, 719-732 |
| HisJ | Histidine | E. coli | —/c | Biochemistry (1994) 33, 4769-4779 |
| (protein: | | | | |
| HBP) | | | | |
| HitA | Iron | Haemophilus influenzae | o/c | Nat. Struct. Biol. (1997) 4, 919-924 |
| | | | | Infect. Immun. (1994) 62, 4515-25 |
| | | | | J. Biol. Chem. (195) 270, 25142-25149 |
| LivJ | leucine/valine/isoleucine | E. coli | —/c | J. Biol. Chem. (1985) 260, 8257-8261 |
| | | | | J. Mol. Biol. (1989) 206, 171-191 |
| LivK | Leucine | E. coli | —/c | J. Biol. Chem. (1985) 260, 8257-8261 |
| (protein: L- | | | | J. Mol. Biol. (1989) 206, 193-207 |
| BP) | | | | |
| MalE | maltodextrine/maltose | E. coli | o/c | Structure (1997) 5, 997-1015 |
| (protein: | | | | J. Bio.l Chem. (1984) 259, 10606-13 |
| MBP) | | | | |
| MglB | glucose/galactose | E. coli | —/c | J. Mol. Biol. (1979) 133, 181-184 |
| (protein: | | | | Mol. Gen. Genet. (1991) 229, 453-459 |
| GGBP) | | | | |
| ModA | molybdate | E. coli | —/c | Nat. Struct. Biol. (1997) 4, 703-707 |
| | | | | Microbiol. Res. (1995) 150, 347-361 |
| MppA | L-alanyl-gamma-D-glutamyl-meso- | E. coli | | J. Bacteriol. (1998) 180, 1215-1223 |
| | diaminopimelate | | | |
| NasF | nitrate/nitrite | Klebsiella oxytoca | —/— | J. Bacteriol. (1998) 180, 1311-1322 |
| NikA | Nickel | E. coli | —/— | Mol. Microbiol. (1993) 9, 1181-1191 |
| opBC | Choline | Bacillus subtilis | —/— | Mol. Microbiol. (1999) 32, 203-216 |
| OppA | oligopeptide | Salmonella typhimurium | o/c | Biochemistry (1997) 36, 9747-9758 |
| | | | | Eur. J. Biochem. (1986) 158, 561-567 |
| PhnD | alkylphosphonate | E. coli | —/— | J. Biol. Chem. (1990) 265, 4461-4471 |
| PhoS (Psts) | phosphate | E. coli | —/c | J. Bacteriol. (1984) 157, 772-778 |
| | | | | Nat. Struct. Biol. (1997) 4, 519-522 |
| PotD | putrescine/spermidine | E. coli | —/c | J. Biol. Chem. (1996) 271, 9519-9525 |
| PotF | polyamines | E. coli | —/c | J. Biol. Chem. (1998) 273, 17604-17609 |
| ProX | Betaine | E. coli | | J. Biol. Chem. (1987) 262, 11841-11846 |
| rbsB | Ribose | E. coli | o/c | J. Biol. Chem. (1983) 258, 12952-6 |
| | | | | J. Mol. Biol. (1998) 279, 651-664 |
| | | | | J. Mol. Biol. (1992) 225, 155-175 |
| SapA | Peptides | Salmonella typhimurium | —/— | EMBO J. (1993) 12, 4053-4062 |
| Sbp | Sulfate | Salmonella typhimurium | —/c | J. Biol. Chem. (1980) 255, 4614-4618 |
| | | | | Nature (1985) 314, 257-260 |
| TauA | Taurin | E. coli | —/— | J. Bacteriol. (1996) 178, 5438-5446 |
| TbpA | Thiamin | E. coli | —/— | J. Biol. Chem. (1998) 273, 8946-8950 |
| TctC | tricarboxylate | Salmonella typhimurium | —/— | |
| ThuE | Trehalose/maltose/sucrose | Sinorhizobium meliloti | —/— | J. Bacteriol. (2002) 184, 2978-2986 |
| TreS | Trehalose | Sulfolobus solfataricus | —/— | Mol. Microbiol. (2001) 39, 1494-1503 |
| tTroA | Zinc | Treponema pallidum | —/c | Gene (1997) 197, 47-64 |
| | | | | Nat. Struct. Biol. (1999) 6, 628-633 |
| UgpB | sn-glycerol-3-phosphate | E. coli | —/— | Mol. Microbiol. (1988) 2, 767-775 |
| XylF | Xylose | E. coli | —/— | Receptors Channels (1995) 3, 117-128 |
| YaeC | Unknown | E. coli | —/— | J Bacteriol (1992) 174, 8016-22 |
| | | | | NCBI accession P28635 |
| YbeJ (GltI) | Glutamate/aspartate (putative, | E. coli | —/— | NCBI accession E64800 |
| | superfamily: lysine-arginine-ornithine- | | | |
| | binding protein) | | | |
| YdcS | (putative, spermidine) | E. coli | —/— | NCBI accession P76108 |
| (b1440) | | | | |
| YehZ | Unknown | E. coli | —/— | NCBI accession AE000302 |
| YejA | (putative, homology to periplasmic | E. coli | —/— | NCBI accession AAA16375 |
| | oligopeptide-binding protein - | | | |
| | Helicobacter pylori) | | | |
| YgiS | oligopeptides | E. coli | —/— | NCBI accession Q46863 |
| (b3020) | | | | |
| YhbN | Unknown | E. coli | —/— | NCBI accession P38685 |
| YhdW | (putative, amino acids) | E. coli | —/— | NCBI accession AAC76300 |
| YliB (b0830) | (putative, peptides) | E. coli | —/— | NCBI accession P75797 |
| YphF | (putative sugars) | E. coli | —/— | NCBI accession P77269 |
| Ytrf | Acetoin | B. subtilis | —/— | J. Bacteriol. (2000) 182, 5454-5461 |
| ZnuA | Zinc | Synechocystis | —/— | J. Mol. Biol. (2003) 333, 1061-1069 |

Bacterial PBPs have the ability to bind a variety of different molecules and nutrients, including sugars, amino acids, vitamins, minerals, ions, metals and peptides, as shown in Table 2. Thus, PBP-based ligand binding sensors may be designed to permit detection and quantitation of any of these molecules according to the methods of the present invention. Naturally occurring species variants of the PBPs listed in Table 2 may also be used, in addition to artificially engineered variants comprising site-specific mutations, deletions or insertions that maintain measurable ligand binding function. Variant nucleic acid sequences suitable for use in the nucleic acid constructs of the present invention will preferably have at least 70, 75, 80, 85, 90, 95, or 99% similarity or identity to the native gene sequence for a given PBP.

Suitable variant nucleic acid sequences may also hybridize to the gene for a PBP under highly stringent hybridization conditions. High stringency conditions are known in the art; see for example Maniatis et al., Molecular Cloning: A Laboratory Manual, 2d Edition, 1989, and Short Protocols in Molecular Biology, ed. Ausubel, et al., both of which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tm) for the specific sequence at a defined ionic strength and pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0M sodium ion, typically about 0.01 to 1.0M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide.

Preferred artificial variants of the sensors of the present invention may exhibit increased or decreased affinity for ligands, in order to expand the range of ligand concentration that can be measured. Artificial variants showing decreased or increased binding affinity for glutamate may) be constructed by random or site-directed mutagenesis and other known mutagenesis techniques, and cloned into the vectors described herein and screened for activity according to the disclosed assays.

In the biosensor nucleic acids of the present invention, fluorescent domains can optionally be separated from the ligand binding domain by one or more flexible linker sequences. Such linker moieties are preferably between about 1 and 50 amino acid residues in length, and more preferably between about 1 and 30 amino acid residues. Linker moieties and their applications are well known in the art and described, for example, in U.S. Pat. Nos. 5,998,204 and 5,981,200, and Newton et al., Biochemistry 35:545-553 (1996). Alternatively, shortened versions of the fluorophores or the binding proteins described herein may be used.

For instance, the present inventors have also found that removing sequences connecting the core protein structure of the binding domain and the fluorophore, i.e., by removing linker sequences and/or by deleting amino acids from the ends of the analyte binding moiety and/or the fluorophores, closer coupling of fluorophores is achieved leading to higher ratio changes. Preferably, deletions are made by deleting at least one, or at least two, or at least three, or at least four, or at least five, or at least eight, or at least ten, or at least fifteen nucleotides in a nucleic acid construct encoding a FRET biosensor that are located in the regions encoding the linker, or fluorophore, or ligand binding domains. Deletions in different regions may be combined in a single construct to create more than one region demonstrating increased rigidity. Amino acids may also be added or mutated to increase rigidity of the biosensor and improve sensitivity. For instance, by introducing a kink by adding a proline residue or other suitable amino acid. Improved sensitivity may be measured by the ratio change in FRET fluorescence upon ligand binding, and preferably increases by at least a factor of 2 as a result of said deletion(s). See provisional application No. 60/658,141, which is herein incorporated by reference in its entirety.

The isolated nucleic acids of the invention may incorporate any suitable donor and acceptor fluorescent protein moieties that are capable in combination of serving as donor and acceptor moieties in FRET. Preferred donor and acceptor moieties are selected from the group consisting of GFP (green fluorescent protein), CFP (cyan fluorescent protein), BFP (blue fluorescent protein), YFP (yellow fluorescent protein), and enhanced variants thereof, with a particularly preferred embodiment provided by the donor/acceptor pair CFP/YFP-Venus, a variant of YFP with improved pH tolerance and maturation time (Nagai, T., Ibata, K., Park, E. S., Kubota, M., Mikoshiba, K., and Miyawaki, A. (2002) A variant of yellow fluorescent protein with fast and efficient maturation for cell-biological applications. Nat. Biotechnol. 20, 87-90). An alternative is the MiCy/mKO pair with higher pH stability and a larger spectral separation (Karasawa S, Araki T, Nagai T, Mizuno H, Miyawaki A. Cyan-emitting and orange-emitting fluorescent proteins as a donor/acceptor pair for fluorescence resonance energy transfer. Biochem J. 2004 381:307-12). Also suitable as either a donor or acceptor is native DsRed from a Discosoma species, an ortholog of DsRed from another genus, or a variant of a native DsRed with optimized properties (e.g. a K83M variant or DsRed2 (available from Clontech)). Criteria to consider when selecting donor and acceptor fluorescent moieties are known in the art, for instance as disclosed in U.S. Pat. No. 6,197,928, which is herein incorporated by reference in its entirety.

As used herein, the term “fluorophore variant” is intended to refer to polypeptides with at least about 70%, more preferably at least 75% identity, including at least 80%, 90%, 95% or greater identity to native fluorescent molecules. Many such variants are known in the art, or can be readily prepared by random or directed mutagenesis of native fluorescent molecules (see, for example, Fradkov et al., FEBS Lett. 479:127-130 (2000)).

The invention further provides vectors containing isolated nucleic acid molecules encoding the improved biosensor genes as disclosed herein. Exemplary vectors include vectors derived from a virus, such as a bacteriophage, a baculovirus or a retrovirus, and vectors derived from bacteria or a combination of bacterial sequences and sequences from other organisms, such as a cosmid or a plasmid. Vectors may be adapted for function in a prokaryotic cell, such as E. coli or other bacteria, or a eukaryotic cell, including yeast, plant and animal cells. For instance, the vectors of the invention will generally contain elements such as an origin of replication compatible with the intended host cells, one or more selectable markers compatible with the intended host cells and one or more multiple cloning sites. The choice of particular elements to include in a vector will depend on factors such as the intended host cells, the insert size, whether) regulated expression of the inserted sequence is desired, i.e., for instance through the use of an inducible or regulatable promoter, the desired copy number of the vector, the desired selection system, and the like. The factors involved in ensuring compatibility between a host cell and a vector for different applications are well known in the art.

Preferred vectors for use in the present invention will permit cloning of the ligand binding domain or receptor genetically fused to nucleic acids encoding donor and acceptor fluorescent molecules, resulting in expression of a chimeric or fusion protein comprising the ligand binding domain genetically fused to donor and acceptor fluorescent molecules. Exemplary vectors include the bacterial pRSET-FLIP derivatives disclosed in Fehr et al. (2002) (Visualization of maltose uptake in living yeast cells by fluorescent nanosensors. Proc. Natl. Acad. Sci. USA 99, 9846-9851), which is herein incorporated by reference in its entirety. Methods of cloning nucleic acids into vectors in the correct frame so as to express fusion proteins are well known in the art.

The invention also includes host cells transfected with a vector or an expression vector of the invention, including prokaryotic cells, such as E. coli or other bacteria, or eukaryotic cells, such as yeast cells, plant cells or animal cells. In another aspect, the invention features a transgenic non-human animal having a phenotype characterized by expression of the nucleic acid sequence coding for the expression of the biosensor. The phenotype is conferred by a transgene contained in the somatic and germ cells of the animal, which may be produced by (a) introducing a transgene into a zygote of an animal, the transgene comprising a DNA construct encoding the biosensor; (b) transplanting the zygote into a pseudopregnant animal; (c) allowing the zygote to develop to term; and (d) identifying at least one transgenic offspring containing the transgene. The step of introducing of the transgene into the embryo can be by introducing an embryonic) stem cell containing the transgene into the embryo, or infecting the embryo with a retrovirus containing the transgene. Transgenic animals of the invention include transgenic C. elegans and transgenic mice and other animals.

Transgenic plants expressing the nucleic acids described herein are also included in the present invention. Transgenic crops include, for example, tobacco, sugar beet, soy beans, beans, peas, potatoes, rice or maize. The expression of genes in dicotyledonous and monocotyledonous plants can be achieved by a variety of procedures known and routinely applied. See, e.g., Potrykus, 1990, Gene transfer methods for plants and cell cultures, Ciba Found. Symp. 154: 198-208. One example is transformation of plants cells with a T-DNA containing the gene of interest using Agrobacterium tumefaciens or Agrobacterium rhizogenes as a means of transformation. For the use of Agrobacterium for the introduction of a gene into a plant cell, the respective gene should be cloned into a binary vector. A variety of different cloning vectors is available for) expression of genes in higher plants using Agrobacterium, e.g. mini binary vectors (Xiang et al., 1999: a mini binary vector series for plant transformation, Plant. Mol. Biol. 40(4): 711-7) and vectors of the pPZP series (Hajdukiewicz et al., 1994, The small, versatile pPZP family of Agrobacterium binary vectors for plant transformation, Plant. Mol. Biol. 25(6): 989-94). Binary plant transformation vectors can replicate in E. coli as well as in Agrobacterium and contain selection markes for selection of transformed plants. For the transfer of the T-DNA, infection of the plant by Agrobacterium is necessary; this can be by infection of leaf pieces, roots, protoplasts, suspension cultures, or flowers of whole plants. For the transformation of Arabidopsis plants, a dipping method is most commonly used (Clough and Bent, 1998, Floral dip: a simplified method for Agrobacterium-mediated transformation of Arabidopsis thaliana, Plant J. 16(6): 735-43). Transformed plants are then selected for resistance against the selection marker, e.g. kanamycin, hygromycin, gluphosinate.

Besides transformation using Agrobacteria, there are many other techniques available for the expression of genes in a plant host cell. These techniques include the fusion or transformation of protoplasts, microinjection of DNA and electroporation, as well as ballistic methods and virus infection. From the transformed plant material, whole plants can be regenerated in a suitable medium, which contains antibiotics or biocides for selection. No special demands are required for plasmid injection and electroporation. Simple plasmids, such as, e.g., pUC-derivatives can be used. Should, however, whole plants be regenerated from such transformed cells, the presence of a selectable marker gene is necessary.

The present invention also encompasses isolated biosensor molecules having the properties described herein, particularly PBP-based fluorescent indicators. Such polypeptides are preferably recombinantly expressed using the nucleic acid constructs described herein. The) expressed polypeptides can optionally be produced in and/or isolated from a transcription-translation system or from a recombinant cell, by biochemical and/or immunological purification methods known in the art. The polypeptides of the invention can be introduced into a lipid bilayer, such as a cellular membrane extract, or an artificial lipid bilayer (e.g. a liposome vesicle) or nanoparticle.

The present invention includes methods of detecting changes in the levels of ligands in samples, comprising (a) providing a cell expressing a nucleic acid encoding an improved sensor according to the present invention and a sample comprising said ligand; and (b) detecting a change in FRET between said donor fluorescent protein moiety and said acceptor fluorescent protein moiety, wherein a change in FRET between said donor moiety and said acceptor moiety indicates a change in the level of said ligand in the sample. The ligand may be any suitable ligand for which a fused FRET biosensor may be constructed, including any of the ligands described herein. Preferably the ligand is one recognized by a PBP, and more preferably a bacterial PBP, such as those included in Table 2 and homologues and natural and artificial variants thereof.

FRET may be measured using a variety of techniques known in the art. For instance, the step of determining FRET may comprise measuring light emitted from the acceptor fluorescent protein moiety. Alternatively, the step of determining FRET may comprise measuring light emitted from the donor fluorescent protein moiety, measuring light emitted from the acceptor fluorescent protein moiety, and calculating a ratio of the light emitted from the donor fluorescent protein moiety and the light emitted from the acceptor fluorescent protein moiety. The step of determining FRET may also comprise measuring the excited state lifetime of the donor moiety or) anisotropy changes (Squire A, Verveer P J, Rocks O, Bastiaens P I. J Struct Biol. 2004 July; 147(1):62-9. Red-edge anisotropy microscopy enables dynamic imaging of homo-FRET between green fluorescent proteins in cells.). Such methods are known in the art and described generally in U.S. Pat. No. 6,197,928, which is herein incorporated by reference in its entirety.

The amount of ligand in a sample can be determined by determining the degree of FRET. First the sensor must be introduced into the sample. Changes in ligand concentration can be determined by monitoring FRET changes at time intervals. The amount of ligand in the sample can be quantified for example by using a calibration curve established by titration in vivo. The sample to be analyzed by the methods of the invention may be contained in vivo, for instance in the measurement of ligand transport on the surface of cells, or in vitro, wherein ligand efflux) may be measured in cell culture. Alternatively, a fluid extract from cells or tissues may be used as a sample from which ligands are detected or measured.

Methods for detecting ligands as disclosed herein may be used to screen and identify compounds that may be used to modulate ligand receptor binding. In one embodiment, among others, the invention comprises a method of identifying a compound that modulates binding of a ligand to a receptor, comprising (a) contacting a mixture comprising a cell expressing a biosensor nucleic acid of the present invention and said ligand with one or more test compounds; and (b) determining FRET between said donor fluorescent domain and said acceptor fluorescent domain following said contacting, wherein increased or decreased FRET following said contacting indicates that said test compound is a compound that modulates ligand binding. The term “modulate” generally means that such compounds may increase or decrease or inhibit the interaction of a ligand with the ligand binding domain.

The methods of the present invention may also be used as a tool for high throughput and high content drug screening. For instance, a solid support or multiwell dish comprising the biosensors of the present invention may be used to screen multiple potential drug candidates simultaneously. Thus, the invention comprises a high throughput method of identifying compounds that modulate binding of a ligand to a receptor, comprising (a) contacting a solid support comprising at least one biosensor of the present invention, or at least one cell expressing a biosensor nucleic acid of the present invention, with said ligand and a plurality of test compounds; and (b) determining FRET between said donor fluorescent domain and said acceptor fluorescent domain following said contacting, wherein increased or decreased FRET following said contacting indicates that a particular test compound is a compound that modulates ligand binding.

The targeting of the sensor to the outer leaflet of the plasma membrane is only one embodiment of the potential applications. It demonstrates that the nanosensor can be targeted to a specific compartment. Alternatively, other targeting sequences may be used to express the sensors in other compartments such as vesicles, ER, vacuole, etc.

It is possible to use the sensors as tools to modify ligand binding, for instance, by introducing them as artificial ligand scavengers presented on membrane or artificial lipid complexes. Artificial ligand scavengers may be used to manipulate signal transduction and the response of cells to various ligands.

The following examples are provided to describe and illustrate the present invention. As such, they should not be construed to limit the scope of the invention. Those in the art will well appreciate that many other embodiments also fall within the scope of the invention, as it is described hereinabove and in the claims.

### EXAMPLES

### Example 1

### Use of Plants Suppressed in Gene Silencing Prevents Silencing of Direct Repeat Transgene

Repeated attempts to express biosensor transgenes in planta led to low or no stable expression. Several independent attempts to generate plants stably expressing biosensors for glucose, maltose and glutamate were not successful, and resulted in either no expression at all or only expression in young plants or expression only in guard cells. However, high expression in all tissues throughout plant development is desired.

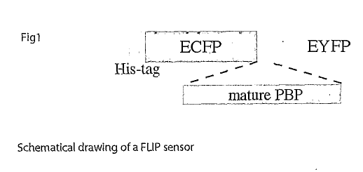

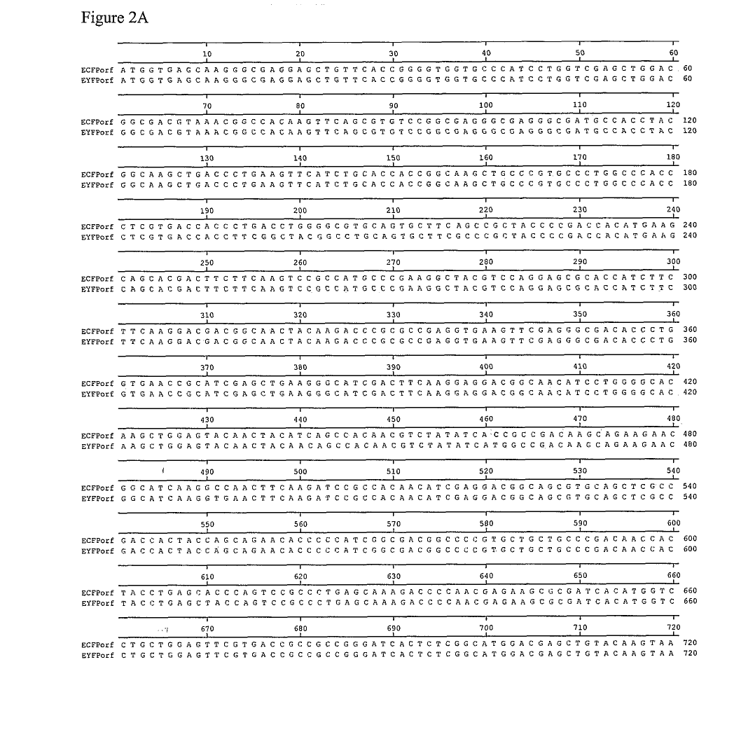

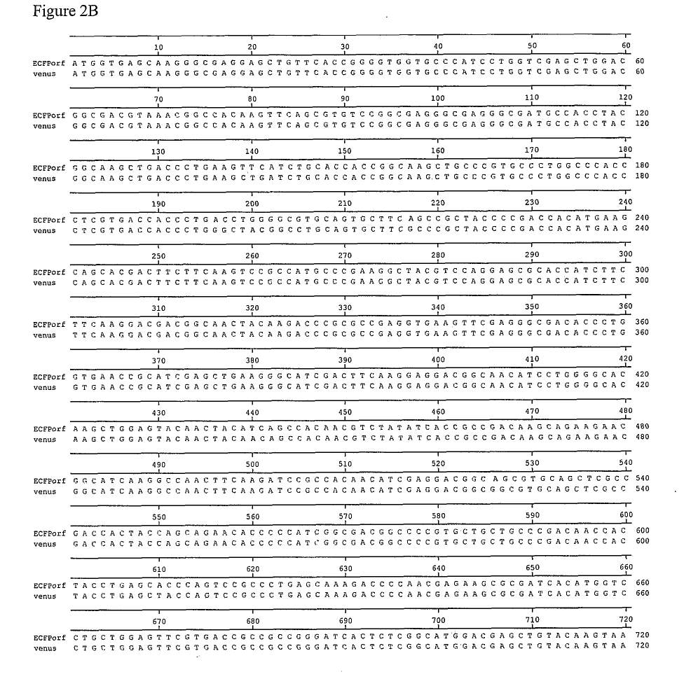

Upon encountering difficulty in expressing the periplasmic binding protein-based biosensors in plants, the present inventors hypothesized that gene silencing in plants was affecting the expression of the transgene constructs via repeat-induced silencing. The biosensors used contain eCFP and eYFP attached to the two ends of a substrate binding protein (FIG. 1). eCFP and eYFP are highly homologous, with only 9 out of 239 amino acids differing on the protein level and 16 out of 720 base pairs differing on the nucleic acid level (FIG. 2A). The use of eYFP Venus (Nagai et al., 2002, A variant of yellow fluorescent protein with fast and efficient maturation for cell biological applications, Nat. Biotech. 20: 87-90) leads to even higher homology, with only 8 amino acids difference at the protein level and 13 base pairs difference at the DNA level (FIG. 2B).

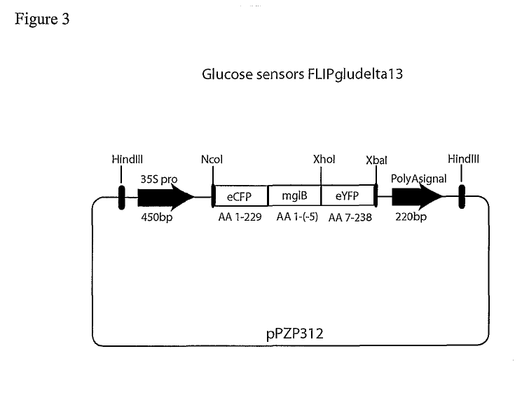

Two Arabidopsis genes, SGS3 and RDR6, have been described as being required for posttranscriptional gene silencing. Peragine et al., 2004, SGS3 and SGS2/SDE1/RDR6 are required for juvenile development and the production of transacting siRNAs in Arabidopsis, Genes and Dev. 18: 2368-79. To test our hypothesis, loss of function mutants for these genes) and Col0 wold type plants were transformed in parallel with the glucose sensor FLIPgludelta 13 (FIG. 3; Deuschle et al., 2005, Construction and optimization of a family of genetically encoded metabolite sensors by semirational protein engineering, Protein Sci. 14:2304-14). sgs3-11 plants were transformed with FLIPglu2μdelta13, rdr6-11 plants were transformed with FLIPglu600μdelta13, and Col0 plants were transformed with FLIPglu2μdelta13 or FLIPglu600μdelta13. For all transformations, the binary vector pPZP312 conferring Basta resistance to transformed plants was used.

Transformants for two different affinity mutants of FLIPgludelta13 (2μ and 600μ) were selected by spraying the seedlings of T1 with BASTA and screened for fluorescence. A higher proportion of the transformants in the sgs3-11 and rdr6-11 mutant background showed fluorescence than in the Col0 background. The fluorescence of the Col0 transformants got weaker with increasing plant age, whereas fluorescence in the sgs3-11/rdr6-11 transformants was at least detectable in plants at the onset of setting seeds (around 30 days after germination). This difference in fluorescence intensity is not likely to be caused by a different number of T-DNA insertions, as segregation of the next generation was around 3:1, suggesting a single insertion for most of the checked plants.

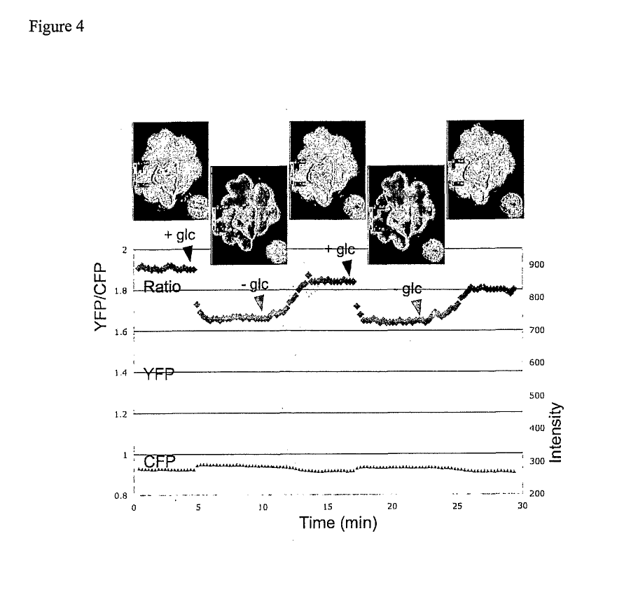

Detection of changes in the cytosolic glucose level of plant cells caused by external application of glucose was possible in rdr6-11 plants expressing FLIPglu600μdelta13 (see FIG. 4). As expected, no cytosolic glucose changes could be observed in sgs3-11 plants expressing FLIPglu2μdelta13, which is most likely saturated in the cytosol of plant cells.

### Example 2

### Decreasing the Homology of Repeat Sequences in Biosensors

In the genetic code, most amino acid sequences are encoded by more than one codon. Exploiting this redundancy, genes can be synthesized using different codons than the original sequence, but still encoding the same amino acid sequence. By changing the codon usage for at least one of the partners of a tandem repeat, the percentage of homology can be significantly decreased.

To circumvent gene silencing of the biosensor constructs, the homology of the eCFP and Venus genes was decreased. To accomplish this, genes encoding a shortened eCFP (amino acids 7-230) and a shortened Venus (amino acids 7-230), each containing different codons with respect to each other while keeping the same amino acid sequences of eCFP and Venus, were synthesized chemically. Shortened versions were synthesized to save on synthesis costs. For cloning into expression vector constructs, the shortened versions may be amplified with extension primers to add back in the terminal sequences, which may also be designed with degenerate substitutions if desired. Alternatively, the shorter versions themselves may be used, as we have found that in some cases the closer coupling of the fluorophores can lead to higher ratio changes upon ligand binding.

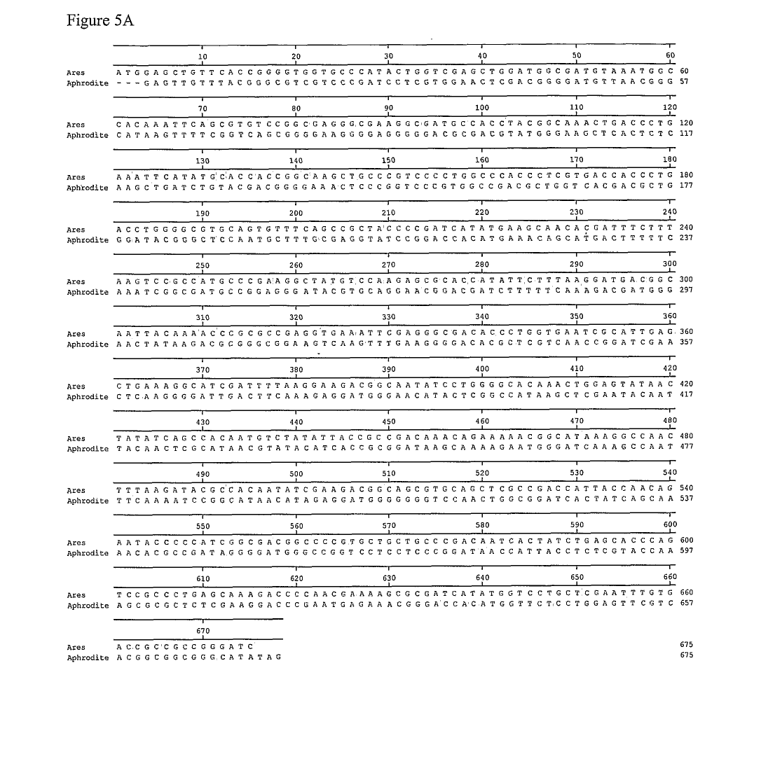

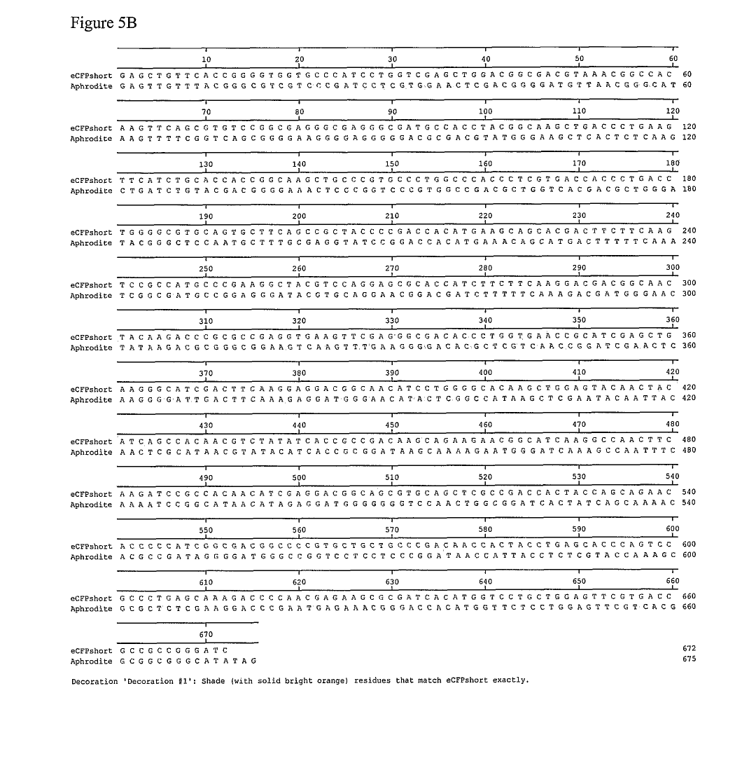

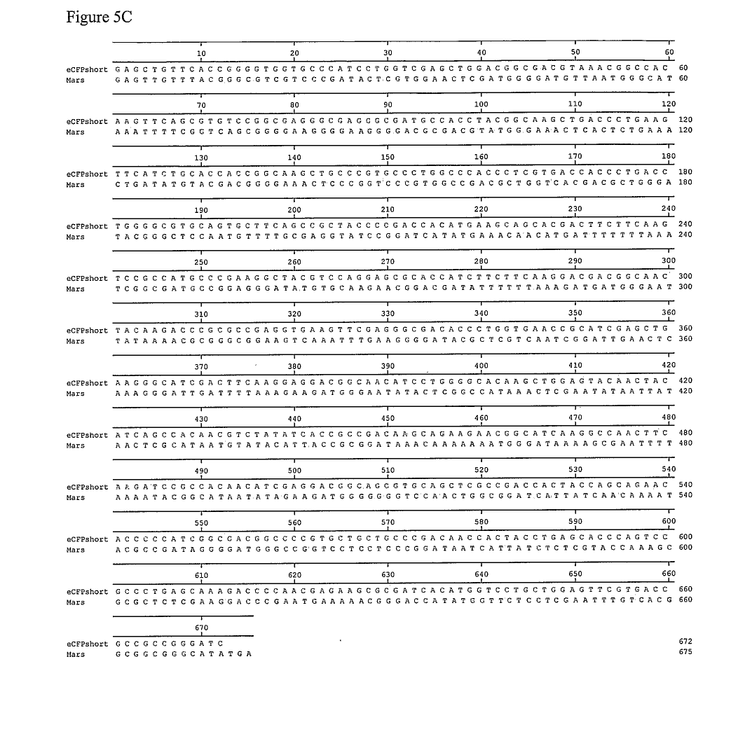

The genetically altered eCFP and Venus sequences were named Ares and Aphrodite, respectively. Roughly every second codon was replaced in each sequence, in an alternating pattern between the two genes. The new sequences differ in 228 out of 672 base pairs, and exclude identical stretches longer than five base pairs (FIG. 5A). If only Venus is replaced by Aphrodite, the longest stretch identical to eCFP is 11 base pairs (FIG. 5B).

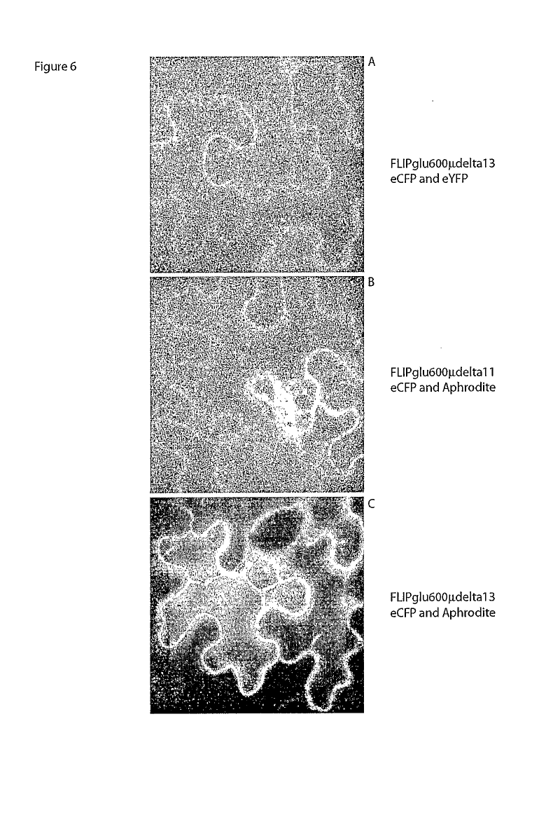

Ares and Aphrodite were used as a FRET pair in FLIPglu600μdelta11 (Deuschle et al., 2005) and successfully expressed in E. coli. Expression of Aphrodite could be shown in plants, where fluorophore expression was visibly enhanced as compared to the eYFP derivative (FIG. 6). Thus, it appears that expression of Ares and Aphrodite in plants should circumvent or at least decrease homology dependent gene silencing. A shortened version of Venus in which nearly every codon was modified was also synthesized and named Mars (SEQ ID NO: 6). Mars is functional as a FRET partner of eCFP in vitro and can be expressed in E. coli. However, Mars has a significantly lower GC content than Aphrodite, which may lead to less than optimal expression in plants.

All publications, patents and patent applications discussed herein are incorporated herein by reference. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed:

1. An isolated nucleic acid which encodes a ligand binding fluorescent indicator, the indicator comprising:
a ligand binding protein moiety;
a donor fluorophore moiety fused to the ligand binding protein moiety; and
an acceptor fluorophore moiety fused to the ligand binding protein moiety;
wherein fluorescence resonance energy transfer (FRET) between the donor moiety and the acceptor moiety is altered when the donor moiety is excited and said ligand binds to the ligand binding protein moiety, and
wherein the nucleic acid sequence encoding at least one of either said donor fluorophore moiety or said acceptor fluorophore moiety has been genetically altered to reduce the level of nucleic acid sequence identity between the nucleic acid encoding the donor fluorophore moiety and the nucleic acid encoding the acceptor fluorophore moiety.

2. The isolated nucleic acid of claim 1, wherein the nucleic acid sequences encoding both of said donor fluorophore moiety and said acceptor fluorophore moiety have been genetically altered to reduce the level of nucleic acid sequence identity between the nucleic acid encoding the donor fluorophore moiety and the nucleic acid encoding the acceptor fluorophore moiety.

3. The isolated nucleic acid of claim 1, wherein said genetic alterations do not change the emission or absorption spectra of said donor fluorophore moiety and said acceptor fluorophore moiety, respectively.

4. The isolated nucleic acid of claim 3, wherein said genetic alterations encode at least one conservative substitution in said donor or acceptor fluorophore.

5. The isolated nucleic acid of claim 3, wherein said genetic alterations comprise at least one degenerate substitution at a wobble position of the donor or acceptor fluorophore coding sequence.

6. The isolated nucleic acid of claim 1, wherein said encoded ligand binding fluorescent indicator demonstrates enhanced function in vivo upon expression of said nucleic acid containing said genetic alterations as compared to said ligand binding fluorescent indicator expressed from said nucleic acid in the absence of said genetic alterations.

7. The isolated nucleic acid of claim 6, wherein said enhanced in vivo function occurs in a plant, animal or fungi.

8. The isolated nucleic acid of claim 6, wherein said enhanced in vivo function is due to a decrease in gene silencing.

9. The isolated nucleic acid of claim 1, wherein said donor and acceptor fluorophore moieties are fused to N- and C-termini of said ligand binding moiety.

10. The isolated nucleic acid of claim 1, wherein at least one of either said donor fluorophore moiety or said acceptor fluorophore moiety is fused to said ligand binding protein moiety at an internal site of said ligand binding protein moiety.

11. The isolated nucleic acid of claim 1, wherein both said donor fluorophore moiety and said acceptor fluorophore moiety are fused to internal sites of said ligand binding protein moiety.

12. The isolated nucleic acid of claim 1, wherein said ligand binding protein moiety is a transporter.

13. The isolated nucleic acid molecule of claim 12, wherein said transporter is selected from the group consisting of channels, uniporters, coporters and antiporters.

14. The isolated nucleic acid of claim 1, wherein said ligand binding protein moiety is a periplasmic binding protein (PBP).

15. The isolated nucleic acid of claim 14, wherein said ligand binding protein moiety is a bacterial periplasmic binding protein.

16. The isolated nucleic acid of claim 14, wherein said donor fluorescent moiety and said acceptor fluorescent moiety are fused to the same lobe of said PBP.

17. The isolated nucleic acid of claim 1, wherein said ligand is an amino acid.

18. The isolated nucleic acid of claim 17, wherein said amino acid is selected from the group consisting of glutamate, aspartate, γ-aminobutyric acid (GABA), aminoacetic acid (glycine) and taurine.

19. The isolated nucleic acid of claim 1, wherein said ligand is a sugar.

20. The isolated nucleic acid of claim 19, wherein said sugar is selected from the group consisting of glucose, galactose, maltose, sucrose, trehalose, arabinose, fructose, xylose, cellobiose and ribose.

21. The isolated nucleic acid of claim 1, wherein said donor fluorophore is selected from the group consisting of a GFP, a CFP, a BFP, a YFP, a dsRED, CoralHue Midoriishi-Cyan (MiCy) and monomeric CoralHue Kusabira-Orange (mKO).

22. The isolated nucleic acid of claim 1, wherein said acceptor fluorophore moiety is selected from the group consisting of a GFP, a CFP, a BFP, a YFP, a dsRED, CoralHue Midoriishi-Cyan (MiCy) and monomeric CoralHue Kusabira-Orange (mKO).

23. The isolated nucleic acid of claim 21, wherein said donor fluorophore moiety is a genetically altered version of eCFP.

24. The isolated nucleic acid of claim 23, wherein said donor fluorophore moiety nucleic acid sequence contains the sequence SEQ ID NO: 1 (Ares).

25. The isolated nucleic acid of claim 1, wherein said acceptor fluorophore moiety is a genetically altered version of YFP VENUS.

26. The isolated nucleic acid of claim 25, wherein said donor fluorophore moiety nucleic acid sequence contains the sequence SEQ ID NO: 2 (Aphrodite).

27. A cell expressing the nucleic acid of claim 1.

28. An expression vector comprising the nucleic acid of claim 1.

29. A cell comprising the vector of claim 28.

30. The expression vector of claim 28 adapted for function in a prokaryotic cell.

31. The expression vector of claim 28 adapted for function in a eukaryotic cell.

32. The cell of claim 29, wherein the cell is a prokaryote.

33. The cell of claim 32, wherein the cell is E. coli.

34. The cell of claim 25, wherein the cell is a eukaryotic cell.

35. The cell of claim 34, wherein the cell is a yeast cell.

36. The cell of claim 34, wherein the cell is an animal cell.

37. The cell of claim 34, wherein said cell is a plant cell.

38. A transgenic animal expressing the nucleic acid of claim 1.

39. A transgenic plant expressing the nucleic acid of claim 1.

40. The isolated nucleic acid of claim 1, further comprising one or more nucleic acid alterations that modify the affinity of the ligand binding protein moiety to said ligand.

41. A ligand binding fluorescent indicator encoded by the nucleic acid of claim 1.

42. A method of detecting changes in the level of a ligand in a sample, comprising:
(a) providing a cell expressing the nucleic acid of claim 1 and a sample comprising said ligand; and
(b) detecting a change in FRET between said donor fluorophore moiety and said acceptor fluorophore moiety,
wherein a change in FRET between said donor moiety and said acceptor moiety indicates a change in the level of said ligand in the sample.

43. The method of claim 42, wherein the step of determining FRET comprises measuring light emitted from the acceptor fluorophore moiety.

44. The method of claim 42, wherein determining FRET comprises measuring light emitted from the donor fluorophore moiety, measuring light emitted from the acceptor fluorophore moiety, and calculating a ratio of the light emitted from the donor fluorophore moiety and the light emitted from the acceptor fluorophore moiety.

45. The method of claim 42, wherein the step of determining FRET comprises measuring the excited state lifetime of the donor moiety.

46. The method of claim 42, wherein said cell is contained in vivo.

47. The method of claim 42, wherein said cell is contained in vitro.

48. The method of claim 42, wherein fluorescence resonance energy transfer (FRET) between the donor moiety and the acceptor moiety is increased when the donor moiety is excited and said ligand binds to the ligand binding protein moiety.

49. The method of claim 42, wherein fluorescence resonance energy transfer (FRET) between the donor moiety and the acceptor moiety is decreased when the donor moiety is excited and said ligand binds to the ligand binding protein moiety.

50. An isolated nucleic acid which comprises a genetically modified fluorophore coding sequence, wherein said genetically modified fluorophore coding sequence contains at least one wobble position base substitution as compared to the fluorophore coding sequence that has not been genetically modified.

51. The isolated nucleic acid of claim 50, wherein said genetically modified fluorophore coding sequence contains at least two wobble position base substitutions as compared to the fluorophore coding sequence that has not been genetically modified.

52. The isolated nucleic acid of claim 50, wherein said genetically modified fluorophore coding sequence contains at least five wobble position base substitutions as compared to the fluorophore coding sequence that has not been genetically modified.

53. The isolated nucleic acid of claim 50, wherein said genetically modified fluorophore coding sequence contains at least ten wobble position base substitutions as compared to the fluorophore coding sequence that has not been genetically modified.

54. The isolated nucleic acid of claim 50, wherein said genetically modified fluorophore coding sequence contains at least fifteen wobble position base substitutions as compared to the fluorophore coding sequence that has not been genetically modified.

55. The isolated nucleic acid of claim 50, wherein said genetically modified fluorophore coding sequence contains at least twenty wobble position base substitutions as compared to the fluorophore coding sequence that has not been genetically modified.

56. The isolated nucleic acid of claim 50, wherein said genetically modified fluorophore coding sequence contains at least thirty wobble position base substitutions as compared to the fluorophore coding sequence that has not been genetically modified.

57. The isolated nucleic acid of claim 50, wherein said genetically modified fluorophore coding sequence contains at least fifty wobble position base substitutions as compared to the fluorophore coding sequence that has not been genetically modified.

58. The isolated nucleic acid of claim 50, wherein said genetically modified fluorophore coding sequence contains at least one hundred wobble position base substitutions as compared to the fluorophore coding sequence that has not been genetically modified.

59. The isolated nucleic acid of claim 50, wherein said fluorophore is a genetically modified version of eCFP.

60. The isolated nucleic acid of claim 59, wherein said fluorophore nucleic acid sequence contains the sequence SEQ ID NO: 1 (Ares).

61. The isolated nucleic acid of claim 50, wherein said fluorophore is a genetically modified version of YFP VENUS.

62. The isolated nucleic acid of claim 61, wherein said fluorophore nucleic acid sequence contains the sequence SEQ ID NO: 2 (Aphrodite).

63. A method of reducing gene silencing of one or more transgenes in a cell, comprising
introducing at least one genetic alteration into said one or more transgenes such that the level of identity in at least one repeat region of said one or more transgenes is reduced, and
transfecting said one or more transgenes into said cell, wherein gene silencing of said one or more transgenes is there by reduced.

64. The method of claim 63, wherein at least two repeat regions are present a single transgene.

65. The method of claim 63, wherein said at least one repeat region is present in two or more different transgenes.

66. The method of claim 63, wherein said at least one repeat region is present in said one or more transgenes and another repeat region is within the DNA of said cell.

67. The method of claim 64, wherein said single transgene is a ligand binding fluorescent indicator comprising a ligand binding protein moiety, a donor fluorophore moiety fused to the ligand binding protein moiety; and an acceptor fluorophore moiety fused to the ligand binding protein moiety.

68. The method of claim 64, wherein said single transgene encodes an artificial single chain dimer.

69. The method of claim 64, wherein said single transgene encodes a protein with duplicated domains (e.g., ABC transporters).

70. The method of claim 65, wherein said two or more different transgenes encode proteins with substantially similar domains.

71. The method of claim 63, wherein said cell is a plant cell.

72. The method of claim 63, wherein said cell is an animal cell.

73. The method of claim 63, wherein said cell is in a plant.

74. The method of claim 63, wherein said cell is in an animal.

75. The method of claim 63, wherein said at least one genetic alteration does not adversely affect the function of the protein encoded by said transgene.

76. The method of claim 75, wherein said at least one genetic alteration encodes a conservative amino acid substitution in said transgene.

77. The method of claim 75, wherein said at least one genetic alteration is a degenerate substitution at a wobble position of said transgene.

78. The method of claim 77, comprising introducing at least two degenerate substitutions at wobble positions of said transgene.

79. The method of claim 77, comprising introducing at least five degenerate substitutions at wobble positions of said transgene.

80. The method of claim 77, comprising introducing at least ten degenerate substitutions at wobble positions of said transgene.

81. The method of claim 77, comprising introducing at least fifteen degenerate substitutions at wobble positions of said transgene.

82. The method of claim 77, comprising introducing at least twenty degenerate substitutions at wobble positions of said transgene.

83. The method of claim 77, comprising introducing at least thirty degenerate substitutions at wobble positions of said transgene.

84. The method of claim 77, comprising introducing at least fifty degenerate substitutions at wobble positions of said transgene.

85. The method of claim 77, comprising introducing at least one hundred degenerate substitutions at wobble positions of said transgene.

86. The method of claim 63, wherein said at least one genetic alteration does not lower the GC content of said transgene.

87. The method of claim 63, wherein said gene silencing is selected from the group consisting of repeat-induced gene silencing (RIGS), repeat-induced point mutation (RIP), paramutation, ectopic trans-inactivation, co-suppression and RNA interference.
